(19) United States
(12) Patent Application Publication
(10) Pub. No.: US 2024/0112760 A1
(43) Pub. Date: Apr. 4, 2024

(54) CHEMICAL SYNTHESIS RECIPE EXTRACTION FOR LIFE CYCLE INVENTORY

(71) Applicant: Microsoft Technology Licensing, LLC, Redmond, WA (US)

(72) Inventors: Kali Diane FROST, Indianapolis, IN (US); Bichlien Hoang NGUYEN, Seattle, WA (US); Jake Allen SMITH, Seattle, WA (US); Yingce XIA, Beijing (CN); Shufang XIE, Beijing (CN); Griffin ADAMS, New York, NY (US); Shang ZHU, Pittsburgh, PA (US)

(73) Assignee: Microsoft Technology Licensing, LLC, Redmond, WA (US)

(21) Appl. No.: 17/937,001

(22) Filed: Sep. 30, 2022

(51) Int. Cl. G16C 20/10; G16C 20/70

(52) U.S. Cl. CPC G16C 20/10; G16C 20/70

(57) ABSTRACT

Examples are disclosed that relate to using natural language processing (NLP) to determine a recipe for a chemical synthesis described in a text to create a life cycle inventory (LCI). One example provides a method comprising receiving an input of a text from a publication comprising a description of a chemical product, and analyzing the text using NLP to determine a recipe for the chemical synthesis, the recipe comprising and action and action metadata, the action metadata comprising a reactant. The method further discloses obtaining LCI information for the reactant, determining an energy utilized for the action, and creating an estimate of an environmental impact for the product.

### BACKGROUND

Life cycle assessment (LCA) is a method for evaluating environmental impacts of a product throughout its entire life cycle. In LCA, production of a given product is broken into a series of process steps called unit processes. A life cycle inventory (LCI) analysis is performed for each unit process to quantify the inputs, outputs, and energy requirements associated with the unit process.

### SUMMARY

Examples are disclosed that relate to using natural language processing (NLP) to determine a recipe for a chemical synthesis described in a text. The determined recipe can then be used to create a life cycle inventory (LCI) for a life cycle analysis (LCA). One disclosed example provides a method for generating an LCI for an LCA. The method comprises receiving an input of a text from a publication comprising a description of a chemical product and analyzing the text using NLP to determine a recipe for the chemical synthesis, the recipe comprising an action and action metadata, the action metadata comprising a reactant. The method further discloses obtaining LCI information for the reactant, determining an energy utilized for the action, and generating an estimate of an environmental impact for the product. The method provides an automated process for creating LCI for chemicals with syntheses that are described in the scientific literature but are not currently in LCA databases, saving time and decreasing costs of performing LCAs.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows a block diagram depicting an example complete life cycle inventory comprising a plurality of life cycle inventories (LCIs) for a life cycle stage.

FIG. 2 shows example details of an LCI of FIG. 1.

FIGS. 3A and 3B show a flow diagram depicting an example method for determining information for an LCI.

FIG. 4 shows an example process flow for determining an LCI from an input of a text comprising information on a chemical synthesis according to the method of FIGS. 3A and 3B.

FIG. 5 shows a flow diagram depicting an example method for extracting one or more recipes from a text that describes one or more chemical syntheses.

FIG. 6 shows an example process flow for determining a proxy chemical for an LCI.

FIG. 7 shows an example computing system with which the method of FIG. 5 or FIG. 6 may be implemented.

FIG. 8 shows a block diagram of an example computing system.

### DETAILED DESCRIPTION

As mentioned above, life cycle assessment (LCA) is a method for evaluating environmental impacts of a product throughout its entire life cycle. In LCA, production of a given product is broken into a series of process steps called unit processes. A life cycle inventory (LCI) analysis is performed for each unit process to quantify the inputs, outputs, and energy requirements associated with the unit process.

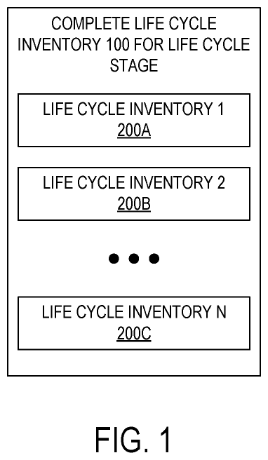

FIG. 1 shows a block diagram depicting a collection of unit process life cycle inventories 200A-C (hereinafter LCIs 200A-C) for a life cycle stage. LCIs 200A-C may be summed to create a complete life cycle inventory 100 for the life cycle stage. as part of an LCA. As one example, LCIs 200A-C may represent manufacturing steps in a manufacturing process.

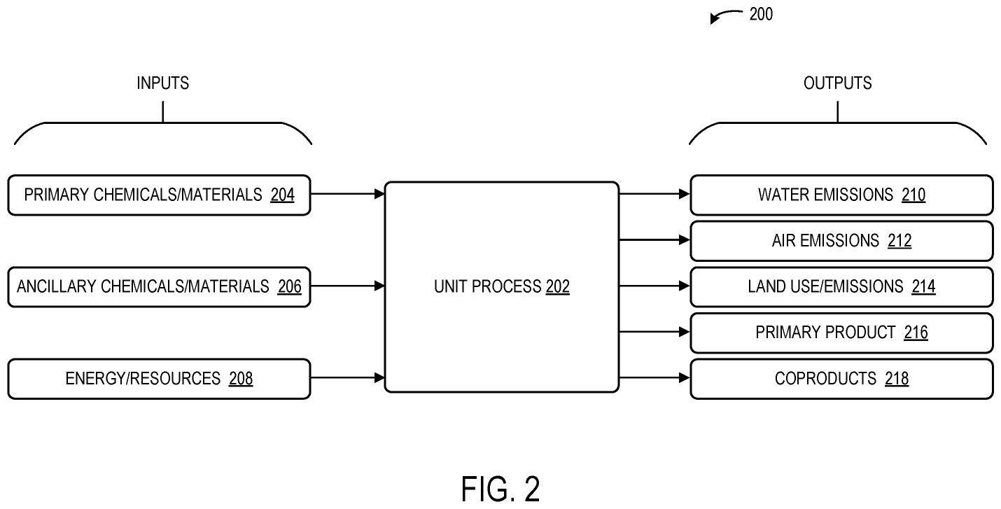

FIG. 2 shows additional details of an LCI 200. LCI 200 may represent an estimated LCI as described below. LCI 200 include inputs and outputs for a discrete unit process 202. Examples of unit processes 202 include manufacturing, mining, usage, transport, purification, refinement, and disposal. LCI 200 may represent any of LCI 1 200A, LCI 2 200B, and/or LCI N 200C. Examples of inputs into unit process 202 include primary chemicals and materials 204, ancillary chemicals and material 206, and energy and resources 208. Examples of outputs from the unit process 202 include water emissions 210, air emissions 212, land use and emissions 214, a primary product 216, and coproducts 218. It will be appreciated that these inputs and outputs presented for the purpose of example, and that any other suitable inputs and outputs may be included in the LCI 200.

For many products, one or more unit processes involve chemical syntheses. However, LCIs for a relatively small number of synthetic processes are available. Increasing the number of LCIs available for synthetic chemicals is difficult and time consuming, even where process knowledge is available. For example, the chemical synthesis literature contains substantial process knowledge from which environmental impacts may be inferred. However, the data in the chemical synthesis literature is heterogeneous and disorganized, thereby impeding the use of chemical synthesis literature for generating LCIs efficiently on a large scale.

Further, inputs and outputs for a relatively small number of synthetic chemicals have been thoroughly quantified. As a result, when determining an LCI for a synthetic chemical, the inputs and outputs of non-quantified chemicals are often estimated using proxy chemicals. However, selection of proxy chemicals by LCA practitioners may be laborious and time-consuming. Furthermore, for a given chemical, selection of a proxy chemical may vary from one LCA practitioner to another.

Accordingly, examples are disclosed that relate to efficiently extracting chemical synthesis data from chemical literature for use in generating LCIs. Briefly, the disclosed examples comprise receiving an input of a text comprising a description of a chemical synthesis of a product. The text is analyzed using natural language processing (NLP) to determine a recipe for the chemical synthesis. The recipe comprises an action and a reactant used in the synthesis, among other possible information. The disclosed examples further comprise obtaining life cycle inventory information for the reactant, determining an energy utilized for the action, and creating a LCI for the product. In this manner, the disclosed examples provide an automated process for determining environmental impacts using chemical synthesis literature. The automated process allows potentially heterogenous and disorganized chemical synthesis data in the chemical synthesis literature to be utilized for large scale LCI generation efficiently. Further, examples also are disclosed that relate to automated proxy chemical selection. The disclosed examples of automating proxy chemical selection may provide for more consistent and efficient proxy chemical selection than manual selection, which otherwise may differ from expert to expert.

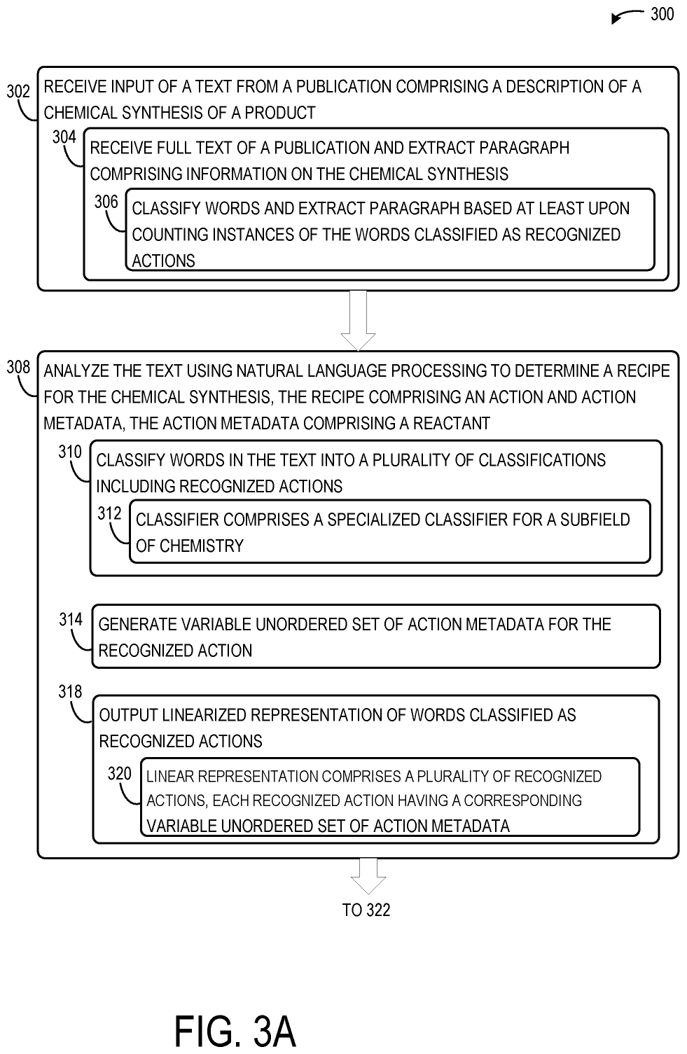

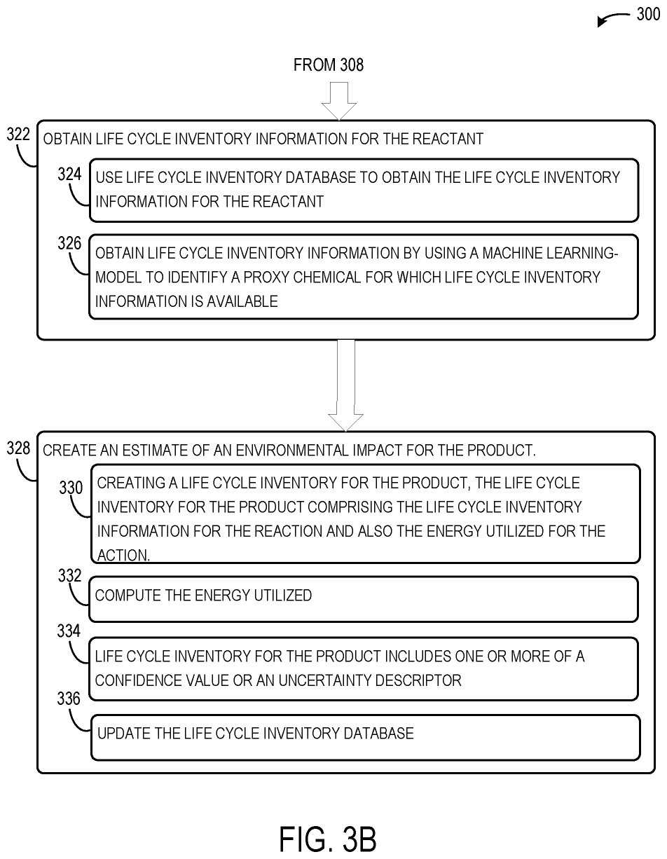

FIGS. 3A and 3B show an example method 300 for determining information for an LCI for inclusion in an LCA as complete life cycle inventory 100 for a life cycle stage. Method 300 may be used, for example, to generate LCI 200. Method 300 comprises, at 302, receiving an input of a text from a publication comprising a description of a chemical synthesis of a product. In some examples, receiving the input of the text at 302 comprises receiving input of a paragraph from a chemical synthesis article that has been extracted from the article prior to input. In other examples, receiving the input at 302 comprises, at 304, receiving a full text of a publication and extracting the paragraphs comprising information on the chemical synthesis. In such examples, any suitable method may be used to extract the paragraph comprising the information on the chemical synthesis.

In some examples, extracting the paragraph comprising information on the chemical synthesis can be performed by classifying words in the text and extracting paragraphs based at least upon counting instances of the words classified as recognized actions, as shown at 306. As a more detailed example, a paragraph can be extracted based upon the paragraph having a threshold number of words classified as recognized actions. Alternatively or additionally, in some such examples a paragraph also can be extracted based upon the paragraph having a specific set of recognized actions as determined from classification. For example, a paragraph may be extracted if it comprises a set of actions representing a sequence of steps in a chemical synthesis (e.g., dissolve, heat, cool, purify, etc.) Example methods for classifying words in a text as recognized actions are described in more detail below in the context of recipe determination.

Method 300 further comprises, at 308, analyzing the text using NLP to determine a recipe for the chemical synthesis. Any suitable NLP methods may be used to determine the recipe. In some examples, as mentioned above with regard to paragraph extraction, words in the text are classified into a plurality of classifications including recognized actions, as indicated at 310. In some such examples, machine learning-based methods can be used to classify the words in the text. As one such example, a machine learning function can be trained to recognize words in texts that describe chemical syntheses that relate to actions used in chemical syntheses. Any suitable machine learning function can be used. In some examples, a neural network can be trained to classify words in texts that disclose chemical syntheses. Such a function can be trained, for example, by inputting texts related to chemical syntheses that include labels for words that represent actions in a chemical synthesis. Such actions may include, as illustrative examples, mix, dissolve, dilute, degas, heat, reflux, cool, recover, filter, rinse, purify, and variants of such words. Such a neural network can be trained using any suitable training methods. As one example, a feed-forward neural network can be trained using training data comprising texts containing labeled words. Labels applied to words can include recognized actions, as well as other classifications. In some examples, other classifications include reactant, product, intermediate, temperature, concentration, volume, mass, and/or other terms related to syntheses. In other examples, classifications may comprise recognized actions and null. Training may be performed, for example, using backpropagation and a suitable cost function, such as a gradient descent function. After training, the trained machine learning function can receive inputs of words from texts that describe chemical syntheses, and classify a word based upon a probability of the word being a recognized action as determined by the machine learning function.

In other examples, a rules-based approach may be used to classify words in a chemical text as a recognized action. In such an example, words within a text that describe a chemical synthesis can be compared to a list of words that represent recognized actions and labeled based upon the word matching a word from the list. In yet other examples, any other suitable approach can be used to label a word in a text as representing a recognized action. In other examples, a recipe may be extracted in any other suitable method than by classifying words as actions.

In some examples, the classifier used at 310 may comprise a generalized classifier that may be used across all fields of chemistry. In other examples, the classifier used at 310 may comprise a specialized classifier for a subfield of chemistry, as shown at 312. A specialized classifier may be trained to identify, or otherwise may utilize, a specialized list of words representing recognized actions that are utilized in a specific subfield of chemistry. As one example, a classifier for organic chemistry may recognize such actions as distill, reflux, precipitate, heat, cool, recover, filter, rinse, purify, and variants thereof, among many other possible words used in organic chemistry syntheses. As another example, a classifier for ceramic syntheses may recognize such actions as calcine, sinter, anneal, grind, mill, and variants thereof, again among many other possible words used in ceramic syntheses. By using specialized classifiers, the natural language processor can identify the actions described in a text for specific subfields of chemistry more efficiently. It will be understood that classifiers for specialized subfields of chemistry may have overlap in the actions recognized.

The recipe can comprise any suitable information about a chemical synthesis in addition to recognized actions. For example, a recipe can include action metadata. Action metadata comprises information associated with a corresponding recognized action. Examples of action metadata can include temperature, volume, molarity, reactant, duration, mass, pressure, and other such parameters related to actions in chemical syntheses. For example, if the recognized action is heat, the action metadata can include one or more reactants to be heated, the temperature to which to heat the reactants, an amount of each reactant to use (e.g., mass), a solvent used for the reaction, a duration for heating the reactant, and rates for heating and cooling.

A recipe may comprise any suitable format In some examples, a recipe may comprise an ordered list of actions (e.g., heat, cool, stir), and for each action, a variable unordered set of metadata such as components, time, and/or temperature. Components can be defined by a name (e.g., magnesium) and associated quantities (e.g., 2 grams). In some such examples a recipe can be generated by a two-step process. A first step comprises extracting spans of text associated with an action. The second step comprises, separately for each action, extracting components, times, and temperatures. As such, in some examples, method 300 generates a variable unordered set of action metadata for the recognized action, as shown at 314. In some examples, the variable unordered set of action metadata can be generated using a left-to-right approach. In a left-to-right approach the words in the texts are analyzed from left to right, and variable unordered sets of action metadata are generated for words that represent recognized actions. In other examples, the variable unordered set of action metadata can be generating using a confidence-first method. In such examples the generation of the variable unordered set of action metadata is based on a confidence value assigned to each word in the text. The confidence can represent the probability that a word is a recognized action. In one example, the generation of a variable unordered set of action metadata for a recognized action may be based at least upon meeting a threshold confidence value.

In some examples, a recipe can comprise a plurality of recognized actions, each recognized action comprising action metadata. In such examples, the recipe may be output as a linearized representation of words classified as recognized actions, as indicated at 318. In some such examples, the linearized representation comprises a plurality of recognized actions, each recognized action having a corresponding variable unordered set of action metadata, as shown at 320. One such illustrative example includes making and recovering a precipitate. In such an example the recognized actions may include heat, mix, cool, and filter. The metadata associated with heat can include a reactant, a solvent, temperature, and duration. The metadata associated with mix can include a reactant, stir bar size, speed of mixing, duration, and temperature. The metadata associated with cool can include temperature and duration. The metadata associated with filter can include duration and vacuum settings. A linearized representation of this example comprises variable unordered sets of action metadata for the recognized actions of heating, mixing, cooling, and filtering, with associated metadata stored for each recognized action.

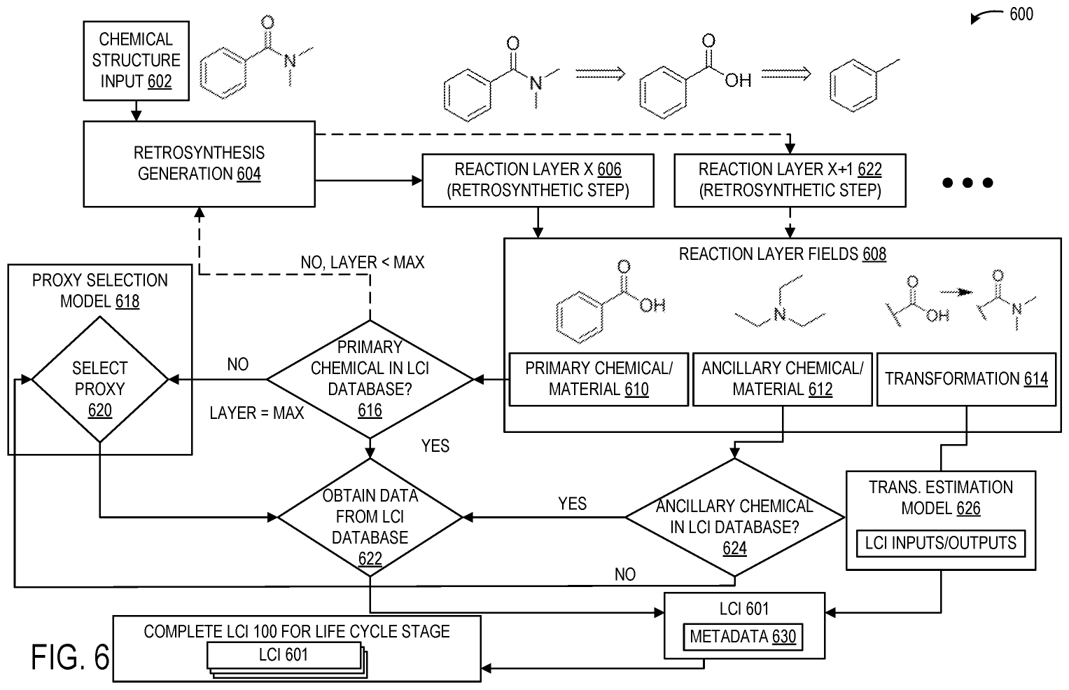

Referring next to FIG. 3B, the recipe can be used to generate LCI information for the chemical synthesis of the product. As such, method 300 further comprises, at 322, obtaining LCI information for the reactant. In some examples, as shown at 324, method 300 uses a LCI database to obtain the life inventory information for the reactant. In other examples, when LCI information is unavailable in the LCI database, method 300 comprises obtaining LCI information by using a machine learning-model to identify a proxy chemical for which LCI information is available, as shown at 326. An example of using a machine-learning model to identify a proxy chemical is described below with regard to FIG. 6. In such examples, LCI information for the proxy chemical selected is obtained to use in creating the LCI. The use of a machine learning-assisted proxy chemical selection model can provide for a more consistent proxy chemical selection when compared to LCA practitioners selecting the proxy chemical.

Method 300 further comprises, at 328, creating an estimate of an environmental impact for the product. In some examples, as indicated at 330, creating the estimate of the environmental impact may comprise creating an LCI for the product, the LCI for the product comprising the LCI information for the reactant and also the energy utilized for the action. Further, at 332, creating the estimate of the environmental impact also comprises determining the energy utilized for the action. In some examples determining the energy utilized for the action comprises calculating the energy using an empirical formula corresponding to the action.

In some examples, method 300 further comprises storing one or more of a confidence value or an uncertainty descriptor with the LCI created, as shown at 334. In some examples, a confidence value can be based upon a probability or probabilities of a classification or classifications determined for recognized actions by a classifier. In other examples, a confidence value alternatively or additionally can be based upon a confidence associated with a proxy chemical selection accuracy. In some examples a confidence value can be generated using an ensemble model for the classifier. In such examples the confidence value is a measure of the confidence of the prediction made by the model. In some such examples, the output of the model may take a form other than a probability. In other examples, a confidence value can be derived from confidence values present in LCIs in databases (e.g., Ecoinvent of Zurich, Switzerland). In such examples the confidence value represents the uncertainty in the underlying data, rather than uncertainty in a model. In further examples, a confidence value can be based upon any other suitable factor or combination of factors. For example, a single confidence value can be derived as a composite of other methods. In some examples, a qualitative uncertainty descriptor can be stored. In other examples, a quantitative uncertainty descriptor can be stored. In further examples, both quantitative and qualitative uncertainty descriptors can be stored. Example qualitative uncertainty descriptors can include geographic coverage, age of the dataset, and representativeness. Example quantitative uncertainty descriptors can include error propagated throughout the LCI generation based on the uncertainty from the chemical synthesis in the text.

In some examples, an LCI may be available in a database, but have a relatively low a confidence value. For example, the LCI may have been generated using a different synthesis, or a different proxy chemical. In such examples, the proxy chemical selection process of 326 may be used to generate an LCI with a potentially higher confidence value, and/or less unfavorable uncertainty information. Thus, at 336, method 300 comprises, after creating the LCI for the product, updating a previously determined LCI in the LCI database.

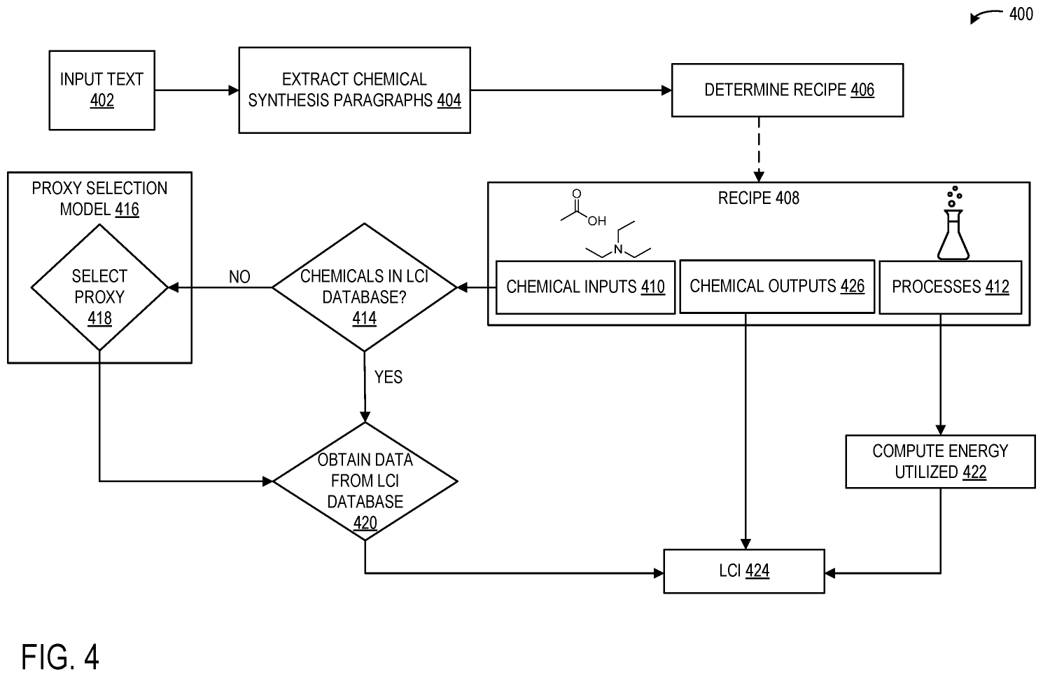

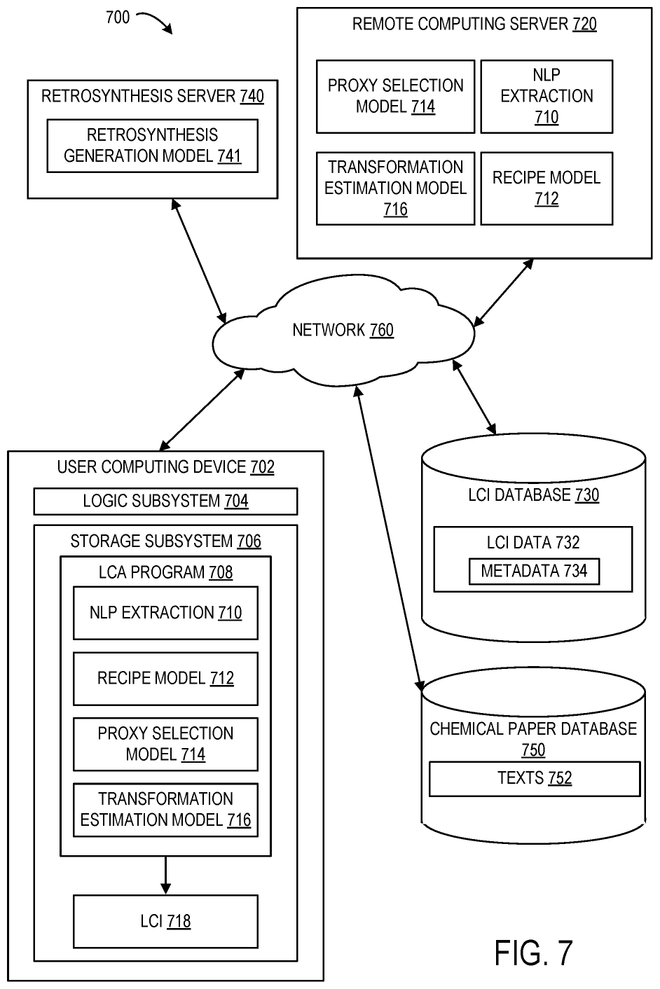

FIG. 4 shows an example method 400 for determining an LCI from an input of a text comprising information on a chemical synthesis generating an LCI implementing method 300. Method 400 may be used, for example, to generate LCI 200. Method 400 is an example implementation of method 300. Method 400 may utilize NLP extraction, machine learning-assisted recipe determination, retrosynthetic analysis, machine learning-assisted proxy chemical selection, and/or machine learning-assisted transformation estimation in determining an LCI. Method 400 may be implemented by any suitable computing system. FIG. 7 shows one example of a suitable computing system 700 comprising a user computing device 702 including an LCI database 730, and an LCA program 708. LCA program 708 is configured to determine LCIs using one or more of NLP extraction, machine learning-assisted recipe determination, retrosynthetic analysis, machine learning-assisted proxy chemical selection, or machine learning-assisted transformation estimation. Other details of FIG. 7 are discussed in more detail below.

Method 400 comprises receiving an input of a text at 402 and extracting chemical synthesis paragraphs from the text at 404, as described above. After extracting the paragraphs, method 400, at 406, determines a synthesis recipe described within the text. The recipe 408 comprises chemical inputs 410, chemical outputs 426, and processes 412. As described with regard to FIG. 3, recipe determination may comprise classifying words in a text as recognized actions and generating sets of action metadata for recognized actions. When a chemical input 410 is available in the LCI database (YES at 414) method 400 comprises obtaining chemical LCI data 420. When the chemical input 410 is not available in the LCI data (NO at 414), proxy selection model 416 is used to select a proxy chemical 418, for which an LCI is available, and obtain proxy chemical LCI data 420. The LCI data obtained at 420 is included in LCI 424. Method 400 further comprises computing the energy utilized 422 for processes 412 from recipe 408. The computed energy is included in LCI 424 along with the LCI data obtained at 420 and the chemical output 426.

In some examples the text from a publication comprising a description of a chemical synthesis of a product can comprise more than one recipe. One example of determining if more than one recipe is in a text can be based on the word count of recognized actions. In other examples semantic analysis of a text may indicate more than one recipe. Examples of semantic analyses include semantic dependency parsing and named entity recognition. In such examples section headings and/or other contextual information can indicate more than one recipe.

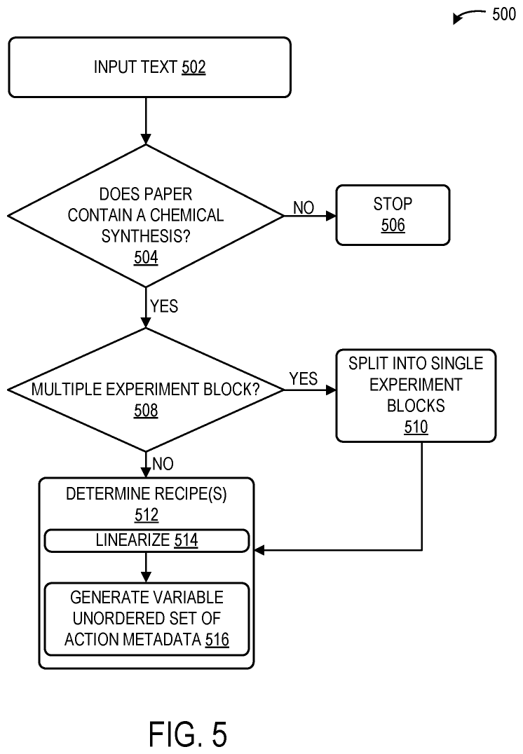

FIG. 5 shows a block diagram of a method 500 for extracting one or more recipes from a text using NLP extraction and a machine learning-assisted recipe model. Method 500 is an example of process 404 in FIG. 4.

Method 500 comprises receiving an input of text 502. Method 500 further comprises determining whether input text 502 contains a description of a chemical synthesis. One example method of determining whether a text contains a chemical synthesis can be determining whether a threshold number of words that represent recognized actions are found in input text 502, as described above. When the text does not contain a chemical synthesis (NO at 504), method 500 stops. On the other hand, when the input text does contain a chemical synthesis (YES at 504), method 500 continues to 508. If the chemical synthesis in input text 502 contains multiple experiment blocks (YES at 508), method 500 splits the experiment blocks into separate single experiment blocks, as shown at 510. Different blocks with different recipes may be determined in any suitable manner. As one example, different paragraphs that meet a threshold count of words representing recognized actions, and that have similar actions with different action metadata, can be considered to describe different recipes. As another example, semantic analysis may be used to identify different syntheses in the text. As yet another example, different recipes may be parsed from a linearized representation of recognized actions in the text. In other words, a determined recipe can be parsed into multiple different recipes.

At 512, method 500 comprising determines the recipe for each identified experimental block. In some examples, determining the recipe at 512 comprises linearizing the actions included in the single experiment block at 514 and generating a variable unordered set of action metadata at 516. The generation of the variable unordered set of action metadata is dependent on the action, such that each action has a corresponding variable unordered set of action metadata.

As mentioned above, when LCI information for a chemical input is not available in an LCI database, a proxy chemical can be selected. Following the selection of a proxy chemical, LCI information for the proxy chemical is used to create an LCI.

FIG. 6 shows a block diagram of an example method 600 for determining an LCI 601 for inclusion in an LCA as complete life cycle inventory 100 for a life cycle stage. LCI 601 is an example of LCI 200. Method 600 may utilize retrosynthetic analysis, machine learning-assisted proxy chemical selection, and/or machine learning-assisted transformation estimation in determining an LCI. The method 600 may be implemented by any suitable computing system. An example is described below with regard to FIG. 7.

Method 600 comprises receiving a chemical structure input 602. The chemical structure input 602 may comprise a structure drawn using a chemical structure drawing program, a chemical name, a unique chemical identifier such a Chemical Abstract Service (CAS) registry number or European Community (EC) number, a simplified molecular-input line-entry system (SMILES) string, or any other suitable form. Chemical structure input 602 corresponds to a reactant from a recipe extracted from a text using NLP. In this example, the chemical structure input comprises N,N-dimethylbenzamide.

Through retrosynthesis generation 604, method 600 is configured to obtain retrosynthetic step data based on chemical structure input 602. The retrosynthetic step data in this example is shown as reaction layer X 606, a retrosynthetic step in which N,N-dimethylbenzamide is formed from benzoic acid. The retrosynthetic step data includes reaction layer fields 608 such as a primary chemical 610, an ancillary chemical 612, and a chemical transformation 614.

Primary chemical 610 comprises a chemical used as a starting material in the retrosynthetic step. In this example, the primary chemical 610 is benzoic acid, the ancillary chemical 612 is triethylamine, and the chemical transformation 614 is an amidation reaction.

When the structure of the primary chemical 610 is not available in the LCI database 730 and no retrosynthetic step data is available for the primary chemical 610 (NO, LAYER=MAX at 616), method 600 comprises inputting the primary chemical 610 into a trained proxy chemical selection model 618 to select a proxy chemical 620 for which an LCI is available and obtain proxy chemical LCI data 622 to include in the LCI 601. Proxy chemicals selected by the proxy chemical selection model 618 have LCIs 200 available in an LCI database and are determined by the proxy chemical selection model 618 to be structurally similar to the primary chemical 610. Further details of the proxy selection model 618 are provided below in relation to description of FIG. 7. An advantage of selecting the proxy chemical 620 from the primary chemical 610 rather than selecting the proxy chemical 620 from the chemical structure input 602 is that the computing system 700 may be more likely to find suitably accurate LCI data.

On the other hand, when the structure of the primary chemical is not available in the LCI database 730 but retrosynthetic step data is available for the primary chemical 610, (NO, LAYER<MAX at 616) method 600 is configured to obtain retrosynthetic step data based on the chemical structure of the primary chemical 610, and determine a chemical structure of an additional primary chemical, namely, the primary chemical for retrosynthetic layer X+1 622, a retrosynthetic step in which benzoic acid is formed from toluene. Although not shown, an additional ancillary chemical (e.g., an oxidizing agent such as potassium permanganate) and an additional chemical transformation (e.g., an oxidation) are also determined in this example. While two reaction layers are shown in FIG. 6, it will be appreciated that three, four, or any number of reaction layers may be generated. In some examples, reaction layers may be generated until either the primary chemical 610 is found in the LCI database 730 (YES at 616), or a maximum number of reaction layers is generated (NO, LAYER=MAX at 616). At 616, “LAYER=MAX” indicates that a reaction layer cannot be generated from the primary chemical 610, for example, because the primary chemical 610 may be structurally too simple for a viable chemical precursor to be available. Further, in some examples, a retrosynthesis algorithm may return a retrosynthesis tree comprising multiple retrosynthesis layers (e.g., all layers for a retrosynthesis in some examples). In such an example, rather than returning to the retrosynthesis algorithm to obtain a next layer of retrosynthesis step data upon finding that a chemical is not available in the LCI database, an additional primary and/or ancillary chemical may be obtained from the retrosynthesis data tree.

As described above, method 600 further comprises determining a chemical structure of an ancillary chemical 612, if any, in the retrosynthetic step data. When the structure of the ancillary chemical 612 is available in an LCI database (YES at 624), the LCA program is configured to obtain chemical LCI data 622 to include in the LCI 601. Method 600 further comprises, when the structure of the ancillary chemical 612 is not available in the LCI database (NO at 624), inputting the ancillary chemical 612 into the trained proxy chemical selection model 618 to obtain a proxy chemical for which an LCI is available, and obtain proxy chemical LCI data to include in the LCI 601.

Continuing with FIG. 6, method 600 is further configured to identify a chemical transformation 614 in the retrosynthetic step data, retrieve LCI data associated with the chemical transformation 614, and include the LCI data associated with the chemical transformation 614 in the LCI 601. Retrieving LCI data for the chemical transformation 614 may be performed by a trained transformation estimation model 626. An example trained transformation estimation model 716 is described in more detail below in relation to FIG. 7. Upon completion of the LCI 601, the LCI 601 may be included in the complete life cycle inventory 100, along with other LCIs in some examples.

In some examples, an LCI 601 generated via method 600 may be stored in an LCI database (e.g., LCI database 730 of FIG. 7). This may allow LCIs generated by method 600 to be retrieved for inclusion in other LCAs. Further, in some examples, metadata 630 related to the creation of an LCI also may be stored for the LCI. Metadata 630 may include, for example, information on how the LCI was generated. Example information includes how many retrosynthetic steps were generated in a retrosynthesis before the chemical of the LCI was output by the retrosynthesis algorithm, and how many proxy chemicals were selected per retrosynthesis step. Such data may be used, for example, to determine an uncertainty metric for the LCI. The uncertainty metric may be represented as a score in some examples. In such an example, LCIs may be rescored as the LCI database is updated with new data. The uncertainty metric may be included in an uncertainty descriptor and/or confidence value, as described above with regard to 334 of FIG. 3.

FIG. 7 shows a block diagram of an example computing system 700. Computing system 700 may implement any of example methods 300, 400, 500, and 600. Computing system 700 includes a user computing device 702, LCI database 730, a retrosynthesis server 740, a remote computing server 720, and a chemical paper database 750. User computing device 702 includes logic subsystem 704 and storage subsystem 706. The NLP extraction 710, the recipe model 712, the proxy selection model 714, and the transformation estimation model 716 are executable by the LCA program 708 on user computing device 702 in order to generate an LCI, such as LCI 424, for inclusion in an LCA as complete life cycle inventory 100 for a life cycle stage. Additionally or alternatively, the NLP extraction 710, the recipe model 712, the proxy selection model 714, and transformation estimation model 716 may be executable by the remote computing server 720, and outputs of these models may be received by user computing device 702.

NLP extraction 710 may comprise any suitable trained machine learning model configured to classify words in a text. In some examples, the trained NLP extraction 710 may comprise a neural network that is trained to classify words comprising text related to chemical synthesis. The neural network may be trained using texts that comprise labels for words that represent recognized actions in a chemical synthesis. In other examples the NLP extraction 710 can comprise a rules-based approach. A rules-based approach can classify words in a text as recognized actions. In such an example, words in a text can be compared to a list of words that represent a recognized action and labeled based upon the word matching a word from the list.

Recipe model 712 may comprise any suitable model for generating a recipe from a text describing a chemical synthesis. In some examples, the trained recipe model may comprise a neural network that is trained with text related to chemical synthesis, as described above with regard to text extraction. The text includes labels for words that represent recognized actions in a chemical synthesis. Recipe model 712 is further comprised to linearize the recognized actions that are extracted from a text. Once recognized actions are linearized recipe model 712 generates a variable unordered set of action metadata for each recognized action. In one example, action metadata can be extracted using NLP extraction 710.

The trained proxy chemical selection model 714 likewise may comprise any suitable trained machine learning function. In some examples, the trained proxy chemical selection model may comprise a neural network that is trained with LCI data contained in a plurality of LCIs stored in LCI data. The LCI data includes, for each of the plurality of LCIs, a chemical structure of a chemical that the LCI describes. Such chemicals also may be referred to herein as possible proxy chemicals. The LCI data may be clustered in the trained proxy chemical selection model based at least upon similarities of the chemical structures of the possible proxy chemicals to one another. The chemical structure of a proxy chemical may be represented by a variety of methods, including a molecular graph in which nodes and edges represent atoms and bonds respectively, a SMILES string, or by a combination of the molecular graph and SMILES string. Other methods for representing a chemical structure of a proxy chemical include encoding the molecular graph M_{g} by a graph neural network (GNN) to output a high-level representation f_{g} or encoding the SMILES string Ms by a transformer to output a high-level representation fₛ. Clustering of the LCI data may be performed by K-Means, K-Medians, Mean-Shift clustering, or any other suitable clustering method. Clustering the LCI data in the trained proxy chemical selection model based at least upon the chemical structure of the proxy chemical may allow for suitably accurate selection of the proxy chemical.

Similarly, the transformation estimation model 716 comprises any suitable trained machine learning function. In some examples, the transformation estimation model comprises a neural network that is trained with LCI data contained in a plurality of LCIs. The LCI data includes at least a starting material, a primary product, and an energy input. The LCI data further includes a reaction representation, the reaction representation being determined based upon a difference between the starting material and the primary product. The LCI data is clustered in the transformation estimation model 716 based at least upon the reaction representation. The reaction representation may be generated by a variety of methods, including a condensed graph of reaction (CGR), a SMILES Arbitrary Target Specification (SMARTS) string, or a combination of CGR and a SMARTS string. Other methods for generating the reaction representation include encoding the CGR R_{g} by a graph neural network (GNN) to output a high-level representation f_{g}′ or encoding the SMARTS string Rs by a transformer to output a high-level representation fₛ′. Clustering of the LCI data may be performed by K-Means, K-Medians, Mean-Shift clustering, or any other suitable clustering method. Clustering the LCI data in the transformation estimation model 626 based at least upon the reaction representation may allow for LCI data associated with the chemical transformation to be accurately selected.

The LCI database 730 includes LCI data 732 for potential proxy chemicals and is accessible by the LCA program 708 of user computing device 702. Potential proxy chemicals include chemicals for which LCI data has been determined, empirically or by other methods. In some such examples, metadata 734 for an LCI comprising information on how the LCI was determined also may be stored. Such metadata may include a score that represents an uncertainty metric in some examples. Additionally or alternatively, LCI data 732 may be stored in the storage subsystem 706 of user computing device 702.

Retrosynthesis server 740 executes a retrosynthesis generation model 741 that performs retrosynthesis generation 604 to generate the reaction layers and reaction layer fields 608. This may be accomplished by algorithms such as those used by commercially available retrosynthetic software. Examples include such software as SYNTHIA™ (MilliporeSigma, Burlington, MA, USA) and IBM RXN (International Business Machines Corporation, Armonk, New York, USA). In some examples, a retrosynthesis program may reside on user computing device 702.

Chemical paper database 750 includes texts 752 that describe chemical syntheses. Chemical paper database 750 is accessible by the LCA program 708 for analyzing texts 752 using NLP to generate recipes for automated LCI determinations. Additionally or alternatively, texts 752 may be stored in the storage subsystem 706 of user computing device 702.

The disclosed examples provide for the automation of LCI creation for chemicals with syntheses that are described in the scientific literature but are not currently in LCA databases. This may decrease times and costs of performing LCAs compared to more manual method.

In some embodiments, the methods and processes described herein may be tied to a computing system of one or more computing devices. In particular, such methods and processes may be implemented as a computer-application program or service, an application-programming interface (API), a library, and/or other computer-program product.

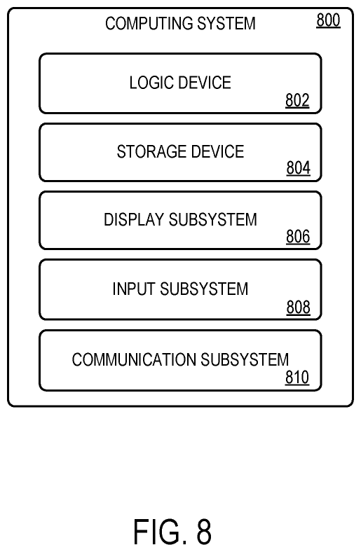

FIG. 8 schematically shows a non-limiting embodiment of a computing system 800 that can enact one or more of the methods and processes described above. Computing system 800 is shown in simplified form. Computing system 800 may take the form of one or more personal computers, server computers, tablet computers, home-entertainment computers, network computing devices, gaming devices, mobile computing devices, mobile communication devices (e.g., smart phone), and/or other computing devices.

Computing system 800 includes a logic device 802 and a storage device 804. Computing system 800 may optionally include a display subsystem 806, input subsystem 808, communication subsystem 810, and/or other components not shown in FIG. 8.

Logic device 802 includes one or more physical devices configured to execute instructions. For example, the logic device may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

Logic device 802 may include one or more processors configured to execute software instructions. Additionally or alternatively, the logic device may include one or more hardware or firmware logic machines configured to execute hardware or firmware instructions. Processors of the logic device may be single-core or multi-core, and the instructions executed thereon may be configured for sequential, parallel, and/or distributed processing. Individual components of the logic device optionally may be distributed among two or more separate devices, which may be remotely located and/or configured for coordinated processing. Aspects of the logic device may be virtualized and executed by remotely accessible, networked computing devices configured in a cloud-computing configuration.

Storage device 804 includes one or more physical devices configured to hold instructions executable by the logic device to implement the methods and processes described herein. When such methods and processes are implemented, the state of storage device 804 may be transformed—e.g., to hold different data.

Storage device 804 may include removable and/or built-in devices. Storage device 804 may include optical memory (e.g., CD, DVD, HD-DVD, Blu-Ray Disc, etc.), semiconductor memory (e.g., RAM, EPROM, EEPROM, etc.), and/or magnetic memory (e.g., hard-disk drive, floppy-disk drive, tape drive, MRAM, etc.), among others. Storage device 804 may include volatile, nonvolatile, dynamic, static, read/write, read-only, random-access, sequential-access, location-addressable, file-addressable, and/or content-addressable devices.

It will be appreciated that storage device 804 includes one or more physical devices. However, aspects of the instructions described herein alternatively may be propagated by a communication medium (e.g., an electromagnetic signal, an optical signal, etc.) that is not held by a physical device for a finite duration.

Aspects of logic device 802 and storage device 804 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The terms “module,” “program,” and “engine” may be used to describe an aspect of computing system 800 implemented to perform a particular function. In some cases, a module, program, or engine may be instantiated via logic device 802 executing instructions held by storage device 804. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

It will be appreciated that a “service”, as used herein, is an application program executable across multiple user sessions. A service may be available to one or more system components, programs, and/or other services. In some implementations, a service may run on one or more server-computing devices.

When included, display subsystem 806 may be used to present a visual representation of data held by storage device 804. This visual representation may take the form of a graphical user interface (GUI). As the herein described methods and processes change the data held by the storage machine, and thus transform the state of the storage machine, the state of display subsystem 806 may likewise be transformed to visually represent changes in the underlying data. Display subsystem 806 may include one or more display devices utilizing virtually any type of technology. Such display devices may be combined with logic device 802 and/or storage device 804 in a shared enclosure, or such display devices may be peripheral display devices.

When included, input subsystem 808 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity.

When included, communication subsystem 810 may be configured to communicatively couple computing system 800 with one or more other computing devices. Communication subsystem 810 may include wired and/or wireless communication devices compatible with one or more different communication protocols. As non-limiting examples, the communication subsystem may be configured for communication via a wireless telephone network, or a wired or wireless local- or wide-area network. In some embodiments, the communication subsystem may allow computing system 800 to send and/or receive messages to and/or from other devices via a network such as the Internet.

Another example provides a method enacted on a computing device. The method comprises receiving input of a text from a publication comprising a description of a chemical synthesis of a product, analyzing the text using natural language processing to determine a recipe for the chemical synthesis, the recipe comprising an action and action metadata, the action metadata comprising a reactant. The method further comprises obtaining life cycle inventory information for the reactant, determining an energy utilized for the action; and creating an estimate of an environmental impact for the product.

In some such examples, creating an estimate of the environmental impact for the product alternatively or additionally comprises creating a life cycle inventory for the product, the life cycle inventory for the product comprising the life cycle inventory information for the reaction and also the energy utilized for the action.

In some such examples, receiving input of the text alternatively or additionally comprises receiving a full text of the publication and extracting a paragraph comprising information on the chemical synthesis.

In some such examples, extracting the paragraph comprising information on the chemical synthesis alternatively or additionally comprises utilizing a rules-based approach.

In some such examples, utilizing the rules-based approach alternatively or additionally comprises using a classifier to classify words in the text into a plurality of classifications including recognized actions, the recognized actions including the action, and extracting the paragraph based at least upon counting instances of the words in the paragraph classified as recognized actions.

In some such examples, analyzing the text to determine the recipe alternatively or additionally comprises using a classifier to classify words in the text into a plurality of classifications including recognized actions, the recognized actions including the action, and outputting a linearized representation of words classified as recognized actions.

In some such examples, the classifier alternatively or additionally comprises a specialized classifier for a subfield of chemistry.

In some such examples, determining the recipe alternatively or additionally comprises generating a variable unordered set of action metadata for the recognized action.

In some such examples, the recognized action in the linearized representation alternatively or additionally comprises a plurality of recognized actions, each recognized action having a corresponding variable unordered set of action metadata.

In some such examples, obtaining the life cycle inventory information for the reactant alternatively or additionally comprises using a life cycle inventory database to obtain the life cycle inventory information for the reactant.

In some such examples, the method alternatively or additionally further comprises, after creating the life cycle inventory for the product, updating a life cycle inventory database.

In some such examples, creating the life cycle inventory for the product alternatively or additionally comprises storing one or more of a confidence value or an uncertainty descriptor.

Another example provides a computing device, comprising a logic subsystem and a storage subsystem holding instructions executable by the logic subsystem to receive input of a text from a publication comprising a description of a chemical synthesis of a product; use natural language processing to extract an action from the text, the action comprising a process in the chemical synthesis, and to extract action metadata regarding a reactant for the process; and based upon the action and the metadata for the action, create a life cycle inventory for the product.

In some such examples, the instructions alternatively or additionally are executable to extract from the text a paragraph comprising information on the chemical synthesis.

In some such examples, the instructions alternatively or additionally are executable to analyze the text to extract the action by using a classifier to classify words in the text into a plurality of classifications including recognized actions related to synthesis, the recognized actions related to synthesis including the action.

In some such examples, the instructions alternatively or additionally are executable to generate a variable unordered set of action metadata for the action.

In some such examples, the instructions are executable to store one or more of a confidence value or an uncertainty description in the life cycle inventory.

Another example provides a method enacted on a computing device, the method comprising receiving input of a text from a publication comprising a description of a chemical synthesis of a product; analyzing the text using natural language processing to determine a recipe for the chemical synthesis, the recipe comprising an action and action metadata, the action metadata comprising a reactant; obtaining life cycle inventory information by using a machine learning model to identify a proxy chemical for which life cycle inventory information is available; determining an energy utilized for the action; and creating a life cycle inventory for the product, the life cycle inventory for the product comprising the life cycle inventory information for the reactant and also the energy utilized for the action.

In some such examples, the method comprises analyzing the text to determine the recipe comprises using a classifier to classify words in the text into a plurality of classifications including recognized actions related to synthesis, the recognized actions related to synthesis including the action.

In some such examples, obtaining the life cycle inventory information for the reactant by using the machine learning model alternatively or additionally comprises applying a retrosynthesis algorithm.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A method enacted on a computing device, the method comprising:
receiving input of a text from a publication comprising a description of a chemical synthesis of a product;
analyzing the text using natural language processing to determine a recipe for the chemical synthesis, the recipe comprising an action and action metadata, the action metadata comprising a reactant;
obtaining life cycle inventory information for the reactant;
determining an energy utilized for the action; and
creating an estimate of an environmental impact for the product.

2. The method of claim 1 wherein creating an estimate of the environmental impact for the product comprises creating a life cycle inventory for the product, the life cycle inventory for the product comprising the life cycle inventory information for the reaction and also the energy utilized for the action.

3. The method of claim 1, wherein receiving input of the text comprises receiving a full text of the publication and extracting a paragraph comprising information on the chemical synthesis.

4. The method of claim 2, wherein extracting the paragraph comprising information on the chemical synthesis comprises utilizing a rules-based approach.

5. The method of claim 3, wherein the rules-based approach comprises using a classifier to classify words in the text into a plurality of classifications including recognized actions, the recognized actions including the action, and extracting the paragraph based at least upon counting instances of the words in the paragraph classified as recognized actions.

6. The method of claim 1, wherein analyzing the text to determine the recipe comprises using a classifier to classify words in the text into a plurality of classifications including recognized actions, the recognized actions including the action, and outputting a linearized representation of words classified as recognized actions.

7. The method of claim 5, wherein the classifier comprises a specialized classifier for a subfield of chemistry.

8. The method of claim 5, wherein determining the recipe includes generating a variable unordered set of action metadata for the recognized action.

9. The method of claim 5, wherein the recognized action in the linearized representation comprises a plurality of recognized actions, each recognized action having a corresponding variable unordered set of action metadata.

10. The method of claim 1, wherein obtaining the life cycle inventory information for the reactant comprises using a life cycle inventory database to obtain the life cycle inventory information for the reactant.

11. The method of claim 1, further comprising, after creating the life cycle inventory for the product, updating a life cycle inventory database.

12. The method of claim 1, wherein creating the life cycle inventory for the product includes storing one or more of a confidence value or an uncertainty descriptor.

13. A computing device, comprising:
a logic subsystem; and
a storage subsystem holding instructions executable by the logic subsystem to receive input of a text from a publication comprising a description of a chemical synthesis of a product;
use natural language processing to extract an action from the text, the action comprising a process in the chemical synthesis, and to extract action metadata regarding a reactant for the process; and
based upon the action and the metadata for the action, create a life cycle inventory for the product.

14. The computing device of claim 13, wherein the instructions are executable to extract from the text a paragraph comprising information on the chemical synthesis.

15. The computing device of claim 13, wherein the instructions are executable to analyze the text to extract the action by using a classifier to classify words in the text into a plurality of classifications including recognized actions related to synthesis, the recognized actions related to synthesis including the action.

16. The computing device of claim 13, wherein the instructions are executable to generate a variable unordered set of action metadata for the action.

17. The computing device of claim 13, wherein the instructions are executable to store one or more of a confidence value or an uncertainty description in the life cycle inventory.

18. A method enacted on a computing device, the method comprising:
receiving input of a text from a publication comprising a description of a chemical synthesis of a product;
analyzing the text using natural language processing to determine a recipe for the chemical synthesis, the recipe comprising an action and action metadata, the action metadata comprising a reactant;
obtaining life cycle inventory information by using a machine learning model to identify a proxy chemical for which life cycle inventory information is available;
determining an energy utilized for the action; and
creating a life cycle inventory for the product, the life cycle inventory for the product comprising the life cycle inventory information for the reactant and also the energy utilized for the action.

19. The method of claim 18, wherein analyzing the text to determine the recipe comprises using a classifier to classify words in the text into a plurality of classifications including recognized actions related to synthesis, the recognized actions related to synthesis including the action.

20. The method of claim 18, wherein obtaining the life cycle inventory information for the reactant by using the machine learning model comprises applying a retrosynthesis algorithm.
